(19) United States
(12) Patent Application Publication
(10) Pub. No.: US 2009/0242609 A1
(43) Pub. Date: Oct. 1, 2009

(54) Vascular Puncture Closure Staple With Tip Protection

(75) Inventor: Glenn Kanner, Plymouth, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/059,482

(22) Filed: Mar. 31, 2008

(51) Int. Cl. A61B 17/068

(52) U.S. Cl. 227/175.1

(57) ABSTRACT

A medical stapling system for closing a vascular puncture includes a mandrel having a flared head that is proximally retracted through a staple in order to splay the legs of the staple. The head has a plurality of channels configured to house the prongs of the staple legs prior to their expansion to prevent physical damage to the tissue and/or the staple tips and to allow the stapling system to have a low delivery profile as the staple is being delivered to the vascular puncture.

### FIELD OF THE INVENTION

The present disclosure relates to systems and methods for closing an opening in a vessel wall after a medical procedure, and in particular, to a system and method for closing a puncture arteriotomy after an intra-luminal procedure such as catheterization.

### BACKGROUND OF THE INVENTION

Various cardiovascular procedures, such as angioplasty, stent placement and atherectomy, require inserting into and manipulating within the vasculature, wires and catheters adapted to perform those procedures. Access to the vasculature typically is through the femoral artery and is percutaneous, involving insertion of a needle in the region of the groin to form a track through subcutaneous tissue and to puncture and create an arteriotomy in the femoral artery. A short guidewire is then advanced through the needle and into the femoral artery. The needle then is removed. An introducer sheath is then advanced over the guidewire, along the track and into the femoral artery. The introducer sheath provides access into the femoral artery, through the arteriotomy, for catheters or other instrumentalities in order to perform the selected procedure.

After the procedure has been completed, the procedural devices are removed and the arteriotomy must be closed. The size of the puncture opening in the artery corresponds to the size of the catheter or percutaneous introducer sheath used, which devices may typically range in diameter from 5 French (1.67 mm) for a diagnostic procedure to 6-10 French (2.00 mm-3.33 mm) for a therapeutic procedure. A number of techniques are known to facilitate closure and healing of the arteriotomy. One technique includes application of pressure at the puncture site for a relatively extended length of time. More particularly, compression has traditionally been applied to the puncture site for at least 30-45 minutes for the wound to close naturally after removal of the catheter. Patients are required to remain decumbent, essentially motionless and often with a heavy sandbag placed on their upper leg, for several hours to ensure that clot has formed at the arteriotomy to prevent bleeding. The recovery time from the medical procedure may be as little as half of an hour, but the recovery time from the wound can exceed 24 hours. This makes wound site management the longer critical care item. The longer the recovery time, the more expensive the procedure becomes, the greater the patient discomfort, and the greater the risk of complications. Other approaches to arteriotomy closure include a compression clamp device, a thrombotic or collagen plug, biological adhesives adapted to seal the arteriotomy, and/or suturing devices.

Medical stapling systems have also been proposed to facilitate closure and healing of the arteriotomy and to resolve some of the concerns associated with arteriotomy closure after vascular catheterization procedures. The medical stapling systems may require blind delivery, viz., without direct visualization, of a staple or clip to gather and hold together sides of the arteriotomy. While the staple is being delivered to the region of the arteriotomy, the prongs of the staple (i.e., the sharp-tipped tissue piercing portions of the staple) may become unintentionally engaged with tissue along the tissue track, and may thus cause damage to the staple or the tissue or both. It would be desirable to provide a staple-type closure system that prevents injury to tissue along the track or physical damage of the staple tips prior to staple deployment. Accordingly, the present invention is directed to such a staple delivery system for closing an arteriotomy.

In addition, it is desirable to provide a staple delivery system with a very low profile. Staple delivery systems typically deliver, or perform the closure modality via a delivery sheath, which holds the tissue track open and may actually enlarge the size of the arteriotomy when delivering the closure element to the target tissue. Accordingly, it is also an object of the present invention to provide a staple delivery system which can be delivered through the tissue track without a delivery sheath, thus preventing incidental enlargement of the arteriotomy. Further, if a delivery sheath is utilized, it is an object of the present invention to provide a staple delivery system having a low profile in order to minimize the size required for the delivery sheath.

### BRIEF SUMMARY OF THE INVENTION

Embodiments of the present invention relate to a staple delivery system for closing an arteriotomy. The staple delivery system includes a tissue staple with at least two staple legs, each staple leg having a distal prong. In addition, the staple delivery system includes a stapler including an elongate mandrel having disposed at its distal end a flared head for expanding the two staple legs when the head is proximally withdrawn through the staple, the head including at least two opposing channels. The prongs are housed within the opposing channels prior to expansion of the staple legs.

In addition, embodiments of the present invention relate to a stapler for closing an arteriotomy. The stapler comprises an outer sleeve, a mandrel slidably received in the outer sleeve, and a head disposed at a distal end of the mandrel, wherein the head is configured to expand the legs of a staple when the mandrel and head are proximally withdrawn through the outer sleeve. The head includes at least two opposing channels adapted for housing distal prongs of the staple legs prior to expansion of the staple legs.

Further, embodiments of the present invention relate to a method of closing an arteriotomy. A stapler is provided, the stapler including a flared head for expanding staple legs of a staple when the head is proximally withdrawn through the staple, wherein the head includes at least two opposing channels. A tissue staple is also provided, the tissue staple including at least two staple legs, each staple leg having a distal prong. The tissue staple is positioned over the head such that at least the prongs are housed within the at least two opposing channels of the head. The staple and the stapler are advanced to the region of the arteriotomy. The head is proximally withdrawn through the staple in order to radially expand at least the prongs of the staple legs. Tissue around the arteriotomy is pierced with the radially expanded prongs, and the prongs are closed inwardly such that the staple gathers tissue around the arteriotomy.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an isometric view of a staple delivery system according to an embodiment of the present invention, with the staple being in its formed or unexpanded configuration.

FIG. 2 is an isometric view of a head of the staple delivery system shown in FIG. 1.

FIG. 3 is an isometric view of a staple of the staple delivery system shown in FIG. 1.

FIG. 4 is an isometric view of the staple delivery system shown in FIG. 1, with the staple being in its expanded or open configuration.

FIG. 5 is a sectional view of body tissue including a vessel with a procedural device and procedural sleeve inserted percutaneously into the vessel lumen.

FIG. 6 is a sectional view of body tissue including a vessel with the procedural device removed from the vessel lumen and the tissue.

FIG. 7 is a sectional view of body tissue including a vessel with the procedural sleeve removed from the vessel lumen and the tissue, with only a guidewire inserted in the vessel.

FIG. 8 is a partial sectional view of a staple delivery system of the present invention being inserted into body tissue.

FIG. 9 illustrates a staple being expanded by a staple delivery system of the present invention at a location just outside of the vessel, adjacent the arteriotomy.

FIG. 10 illustrates the staple deployed by a staple delivery system of the present invention to close the arteriotomy.

FIG. 11 illustrates the staple deployed by the stapler of the present invention, which has been removed from the body tissue.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other body passageways where it is deemed useful. Furthermore, there is no intention to be bound by any expressed or implied theory presented in the preceding technical field, background, brief summary or the following detailed description.

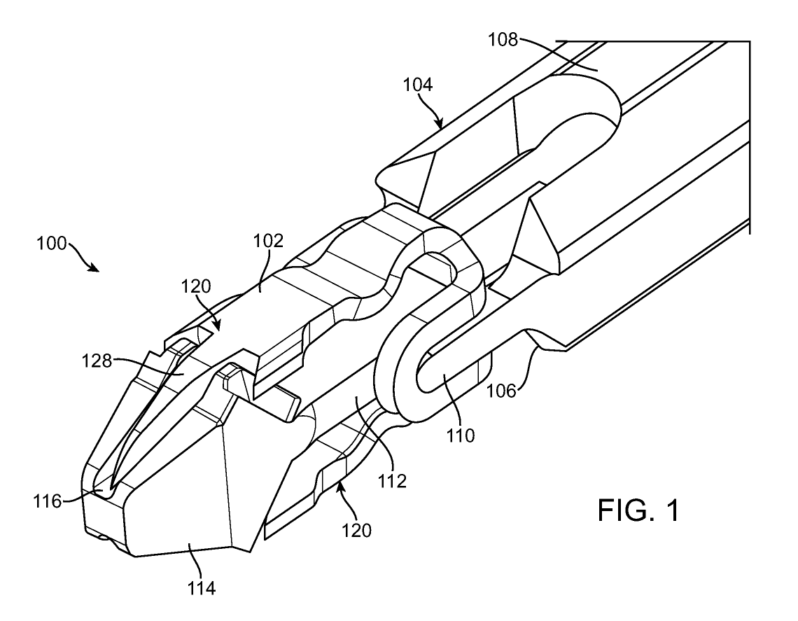

Embodiments of the present invention relate to a device for delivering a closure element that closes an arterial puncture or arteriotomy. More particularly, the present invention relates to a staple delivery system having a head for expanding staple legs of a staple. The head is proximally withdrawn or retracted such that it splays the distally-directed staple legs to an expanded configuration for piercing tissue surrounding the arteriotomy. Further retraction of the head allows the staple legs to resiliently close towards each other, gathering and holding together the tissue surrounding the arteriotomy. The head has opposing channels configured to receive the prongs of the staple legs prior to their expansion or deployment. The channels in the head guard or protect the prongs of the staple legs so that the sharp tips do not become unintentionally engaged with other objects or tissue along the tissue track, and possibly cause damage to the staple or the tissue or both while the staple is being delivered to the region of the arteriotomy. The channels thus prevent damage to the tissue track or the staple tips prior to staple deployment. In addition, the channels have a depth sufficient to house the prongs of the staple legs and thus provide the stapling system with a low delivery profile. Further details and description of the embodiments of the present invention are provided below with reference to FIGS. 1-11.

FIG. 1 is an isometric or perspective view of a staple delivery system 100 in accordance with an embodiment of the present invention. Staple delivery system 100 includes a stapler 104 for delivering a staple 102 to an arterial puncture or arteriotomy. As a general overview, stapler 104 includes a percutaneously placeable tube or sleeve 108 having a distal end 106 for holding and deploying staple legs 120 of staple 102. A distal end of each staple leg 120 includes a tapered portion or prong 128 for piercing tissue surrounding the arteriotomy. The stapler of the present invention guards prongs 128 of staple legs 120 prior to their expansion or deployment. Stapler 104 includes an actuator mechanism at the distal end thereof to cause staple legs 120 of staple 102, or at least prongs 128, to splay or expand outwardly such that staple 102 may pierce and engage tissue broadly about an arteriotomy. Then, the actuator mechanism permits staple 102 to gather or pucker the tissue about the arteriotomy as the staple legs 120 resiliently retract inwardly. The components of the actuator mechanism are movable along an axial direction (e.g., along an inserted guidewire or through a sheath) and extend to the proximal end of the system (not shown) where they may be controlled to perform their respective functions and movements at the distal portion.

A plurality of fingers 110 distally extend from sleeve distal end 106 for receiving staple 102. A mandrel 112 is an elongate rod that is slidable within sleeve 108. Mandrel 112 includes a head 114 at the distal end thereof for generating expansion forces on staple 102 in order to outwardly expand staple legs 120. Head 114 includes a plurality of opposing channels 116 which in use are aligned with prongs 128 of staple legs 120 for housing and guarding at least prongs 128 prior to the expansion or deployment of staple legs 120. Mandrel 112 is movable along an axial direction and extends to the proximal end of staple delivery system 100 where it may be manipulated within sleeve 108 to expand or open staple legs 120 located at the distal portion of staple delivery system 100. Mandrel 112 may be retracted or withdrawn in a proximal direction such that head 114 located at the distal end thereof engages staple legs 120, thereby pushing staple legs 120 apart and expanding staple legs 120 to a configuration suitable to gather and hold together tissue surrounding the arteriotomy.

In one embodiment, mandrel 112 extends through sleeve 108 to a proximally-located handle (not shown) wherein a mechanism may be manually operated to move mandrel 112 within sleeve 108, as will be understood by one of ordinary skill in the art of medical stapling devices. In one embodiment, mandrel 112 and sleeve 108 may each be approximately 30 cm in length. Other actuator mechanisms may also be utilized for effecting the relative motion between head 114 and staple 102. For example, the actuator mechanism may include a cam (not shown) movable in a linear motion. Movement of the cam can be manual or through an electronically controllable motor. The cam would be urged in a proximal direction to cause relative motion between the mandrel and the sleeve, and thus between the head and the staple to force the staple into the expanded or open configuration.

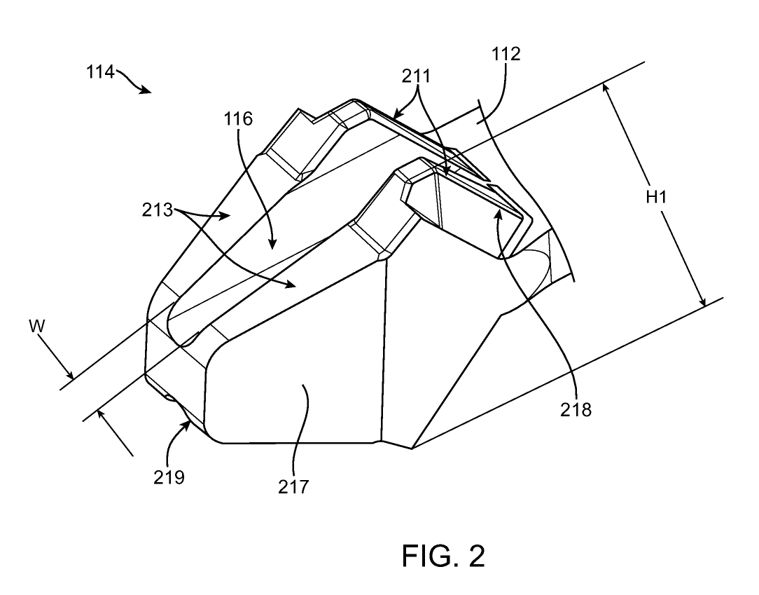

FIG. 2 is an enlarged illustration of head 114. Proximal portion 218 of head 114 includes a plurality of ramps 211 on the top and bottom surfaces of head 114 that flare in a radial direction for expanding staple legs 120 of staple 102 as will be described in further detail below. The term “flare” as used herein is intended to mean that the diameter or transverse dimension of the outer surface of head 114 increases in a distal direction. Note that, whereas ramps 211 are described as being disposed on the “top” and “bottom” of head 114, the expansion of the two staple legs of the current embodiment means the distal tips of the staple legs expand away from the top and bottom of head 114. It will be understood by one of skill in the art that top, bottom and side are terms used only to assist the reader in understanding the relative positions of the components of the disclosure. In addition, although ramps 211 on the “bottom” of head 114 are not visible on FIG. 2, it will be understood that the features shown and described on the visible “top” of head 114 are also present in mirror-image form on the “bottom” of head 114. In the two-legged staple embodiment shown in FIGS. 1-4, the “side” surfaces of proximal portion 218 of head 114 are substantially flat and parallel for being drawn between fingers 110 of sleeve 108. In alternative embodiments of the disclosure wherein the staple has more than two staple legs, the terms describing the head as having “top,” “bottom,” and “sides” would, of course, have somewhat different meanings than those described above. However, the features and functions of the disclosure are still operable in such embodiments. For example, in an embodiment having a four-legged staple, head 114 would have ramps 211 on the “top,” “bottom,” and both “sides” for splaying the corresponding legs of the staple.

Distal portion 219 of head 114 includes a plurality of surfaces 213 on the top and bottom surfaces of the head 114 that taper in a distal direction. The term “taper” as used herein is intended to mean that the diameter or transverse dimension of the outer surface of head 114 decreases in a distal direction. Note that, whereas tapered surfaces 213 are described as being disposed on the “top” and “bottom” of head 114, then closure of the staple legs means the prongs close toward the top and bottom of head 114. It will be understood by one of skill in the art that top, bottom and side are terms used only to assist the reader in understanding the relative positions of the components of the disclosure. In addition, although tapered surfaces 213 on the “bottom” of head 114 are not visible on FIG. 2, it will be understood that the features shown and described on the visible “top” of head 114 are also present in mirror-image form on the “bottom” of head 114. Tapered surfaces 213 of head 114 correspond or align with the shape of prongs 128 in order to provide protective material alongside channels 116 of head 114, therefore guarding or protecting prongs 128 prior to expansion of staple legs 120. Distal portion 219 of head 114 also includes a plurality of surfaces 217 on the sides of the head 114 that taper in a distal direction. Tapered surfaces 217 of head 114, in conjunction with tapered surfaces 213, provide a tapered distal end of system 100 to facilitate advancement of stapler 104 through a narrow tissue track to the region of the arteriotomy. Although tapered surfaces 217 on the “right side” of head 114 are not visible on FIG. 2, it will be understood that the features shown and described on the visible “left side” of head 114 are also present in mirror-image form on the “right side” of head 114.

Channels 116 extend longitudinally from the proximal end to the distal end of head 114. Channels 116 include a width W that is selected to receive prongs 128 of staple legs 120 as will be described in further detail below. In addition, channels 116 have a depth sufficient to completely house prongs 128 of staple legs 120 in order to prevent damage to both the staple legs and the surrounding tissue as the staple is tracked to the region of the arteriotomy. The depth of channels 116 extends at least partially within the material of head 114, and, alternatively, can extend completely through (not shown) head 114. FIG. 2 illustrates head 114 having two opposing channels 116 disposed on the “top” and “bottom” of head 114 for housing a two-pronged staple. Although channel 116 on the “bottom” of head 114 is not visible on FIG. 2, it will be understood that the features shown and described on the visible “top” of head 114 are also present in mirror-image form on the “bottom” of head 114. However, it will be understood by those of ordinary skill in the art that the number of opposing channels provided in head 114 corresponds to the number of staple prongs of the staple. The staple may include any appropriate number of prongs gripping the tissue about the arteriotomy, preferably between two and six prongs. Accordingly, head 114 will include a corresponding number of channels 116 for housing the staple prongs. For example, in an embodiment having a four-legged staple, head 114 would have channels 116 on the “top,” “bottom,” and both “sides” for housing the corresponding prongs of the staple.

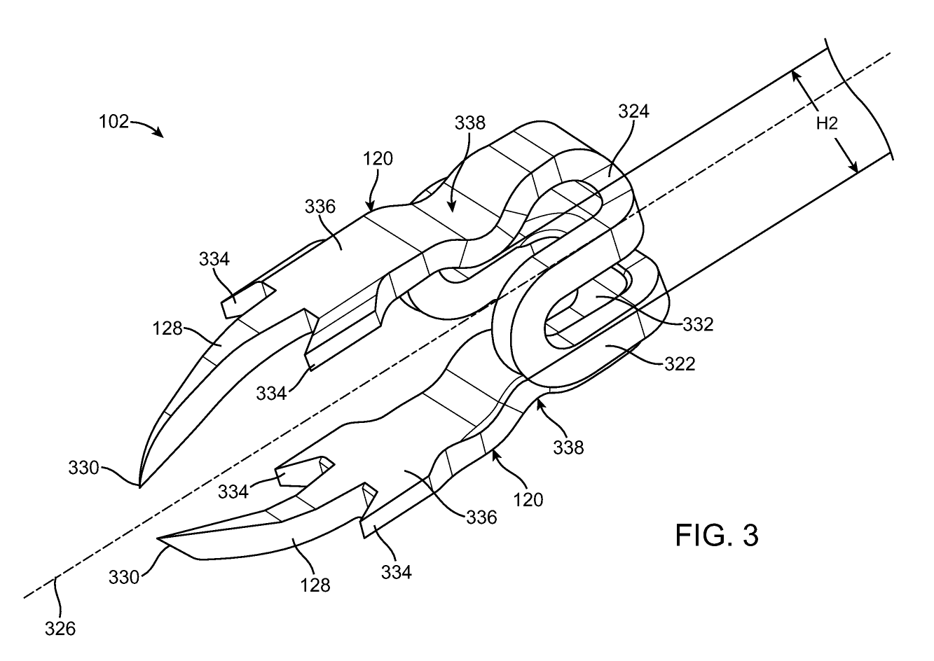

FIG. 3 illustrates staple 102 in its formed, relaxed or static configuration with no forces applied thereto to cause deformation. Staple 102 is provided to close a puncture in an artery or vein following a diagnostic or interventional catheterization procedure. However, it should be apparent to those of ordinary skill in the art that staple 102 may be used for general tissue repair, and is not just limited to vascular repair. Staple 102 comprises a plurality of staple legs 120 and a plurality of generally U-shaped tabs 322, formed between and joining the proximal ends of adjacent staple legs 102 via shoulders 324, the staple legs and tabs being annularly arranged about a centerline or axis 326. Staple legs 120 and tabs 322 extend generally parallel to axis 326. At the distal ends of each staple leg 120, prongs 128 for piercing tissue surrounding the arteriotomy are tapered portions that extend inwardly toward axis 326. A base portion 336 of staple leg 120 extends between each prong 128 and the proximal end of staple 102. Base portion 336 may have a width that is greater than the width of prong 128. Each base portion 336 includes a protrusion 338 that extends inwardly toward axis 326 such as, for example, the inner surface of an indentation in base portion 336. Protrusion 338 is defined at a location sufficiently spaced from a proximal end of base portion 336 to provide leverage for resiliently splaying staple legs 120, as described in detail below. Protrusions 338 are generally depicted as shallow bumps, formed at generally the same location along the length of each staple leg 120, so that protrusions 338 extend toward one another. Protrusions 338 are formed to cooperate with head 114 to expand prongs 128 outwardly, as will become apparent from the description below.

A common portion or shoulder 324 is formed at a proximal portion of each staple leg 120 for connecting tabs 322 and staple legs 120. Each shoulder 324 is common to both a staple leg and a tab and is generally defined by a relatively flat portion generally orthogonal to the axis 326. Shoulders 324 may be viewed as an extension of each staple leg 120, bent inwardly toward axis 326. The general U-shape of tabs 322 defines slots 332 within each tab 322, which are configured to receive fingers 110 of sleeve 108. Although FIG. 3 depicts staple 102 having two staple legs 120, as well as two tabs 322, this should only be considered as being exemplary. It may be desirable to adapt staple 102 with more than two staple legs, with corresponding tabs and shoulders for a given application. Also, it is not necessary that each staple leg is the same length, or that each staple leg has the same overall dimensions.

It may be desirable that staple 102 be deployed into vascular tissue such that staple legs 120 do not fully pierce through the tissue, but rather pierce only deeply enough to grasp and hold the tissue together. For example, for vascular closure applications it may be desirable that prongs 128 not extend into the bloodstream where they might act as sites for thrombus formation, but rather pierce into the tissue and stop short of piercing through the tissue wall. To that end, staple 102 may include one or more tissue stops 334 located along the length of each leg 120, and spaced from each distal tip 330 of staple leg 120 to permit prongs 128 to pierce the surface of the tissue, but to resist prongs 128 piercing all the way through the tissue. In the embodiment shown in FIG. 3, tissue stops 334 are shown adjacent base portion 336. However, the position of tissue stops 334 along the length of staple legs 120 is selected to facilitate tissue grabbing (but not complete tissue piercing) and can vary from application to application.

Stapler 104 expands or opens staple legs 120 outwardly from axis 326 in order to deploy staple 102 about the arteriotomy. It is advantageous to splay staple legs 120 so that insertion of prongs 128 into the tissue occurs at a locus of points separated by a distance greater than the distance separating prongs 128 when staple 102 is in a static configuration. Thus, the splayed staple legs 120 can grasp a large portion of tissue around the wound site, thereby providing a more consistent wound closure by closing the wound with more of the surrounding tissue and promoting complete, or near complete closure of the wound.

Referring back to FIG. 1, staple 102 is shown in a configuration that may be referred to as being closed, relaxed, unexpanded or static, wherein no forces are applied to the staple to cause deformation. In operation, staple 102 is positioned over the outer surface of mandrel head 114 of stapler 104, with mandrel 112 extending proximally along the axis 326 of staple 102 and staple legs 120 pointing distally towards the arteriotomy. Prongs 128 are housed within slots or channels 116 of head 114. As such, prongs 128 are protected from damage while staple 102 is being delivered to the region of the arteriotomy. Slots 332, formed by the generally U-shaped tabs 322 of staple 102, engage fingers 110 of sleeve 108.

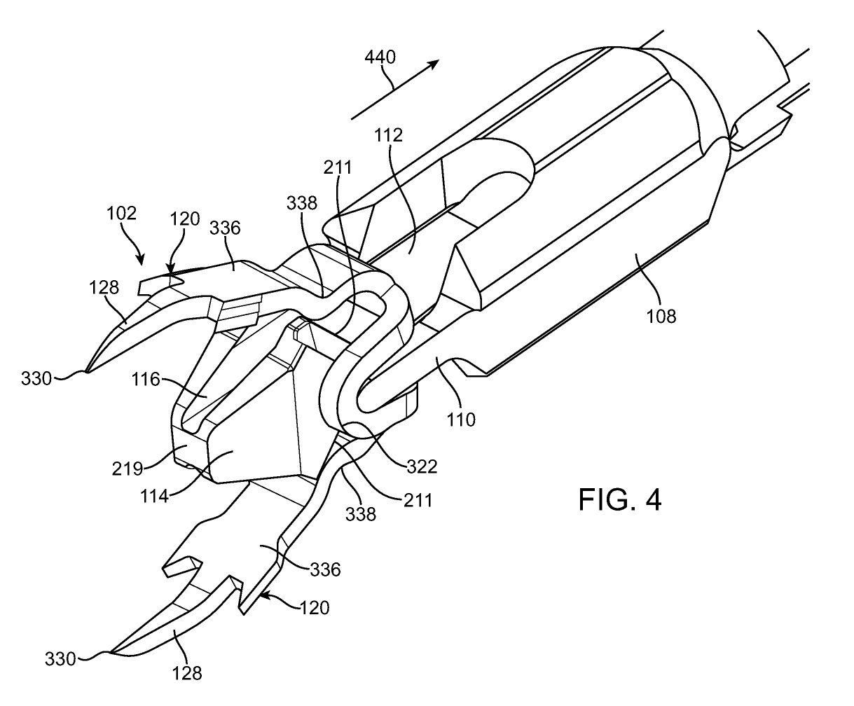

FIG. 4 illustrates the working relationship between staple 102, sleeve 108 and head 114 of mandrel 112. Staple 102 is urged into the open or expanded configuration by the relative movement of head 114 and staple 102. For example, sleeve 108 may provide distally directed force to hold staple 102 stationary while mandrel 112 is pulled proximally within sleeve 108 and staple 102. Alternatively, mandrel 112 may provide proximally directed force to hold staple 102 stationary while sleeve 108 is advanced distally along mandrel 112. In either case, the relative motion between mandrel 112 and sleeve 108 has the same expansive effect on the staple, but either component may be held stationary with respect to the vessel wall during different stages of staple deployment.

Head 114 is retracted in a proximal direction relative to staple 102 (indicated by directional arrow 440) such that ramps 211 on the top and bottom surfaces of head 114 engage protrusions 338 of staple legs 120, and splay staple legs 120 apart to transform staple 102 from the closed configuration (FIGS. 1 and 3) into the open configuration (FIG. 4). Ramps 211 define a maximum external dimension H1 of mandrel head 114. The internal transverse dimension between protrusions 338 of opposing staple legs 120 is H2, where H1>H2. Thus, as mandrel head 114 slides through staple 102, the relatively larger external dimension (H1) of mandrel head 114 causes protrusions 338 to separate, thus forcing prongs 128 to expand outwardly, as depicted in FIG. 4. The amount of expansion is therefore set by the relative dimensions of H1 and H2, and can be adjusted to meet a particular desired result. In the open or expanded configuration, staple legs 120 are resiliently splayed or pivoted around their substantially non-expanding proximal ends, such that distal tips 330 are radially or transversely separated from one another and from central axis 326 of staple 102. Staple legs 120 engage tissue adjacent the arteriotomy (e.g., tissue on opposite sides or surrounding a wound) while staple legs 120 are in the open configuration. Additional details of the working relationship between the staple, the sleeve and the head of the mandrel are further described in U.S. Patent Publication No. 2004/0267312 to Kanner, the entire disclosure of which is incorporated herein by reference.

In one embodiment of the present invention, staple 102 may be constructed from a material that is sufficiently elastic to be resiliently deformed during deployment at the wound site. For example, staple 102 may be constructed out of a spring-type metal such as stainless steel or titanium, or a superelastic metal such as nickel-titanium (nitinol). Staple legs 120 are resiliently biased toward the closed or unexpanded configuration such that when released from stapler 104, staple 102 tends to revert back to the closed or unexpanded configuration illustrated in FIGS. 1 and 3. In the closed or unexpanded configuration, prongs 128 of staple legs 120 extend inwardly toward axis 326.

With staple 102 formed from a resilient metal, staple 102 will tend to revert back to the closed or unexpanded configuration once head 114 is withdrawn proximally of protrusions 338. More particularly, with tissue surrounding the arteriotomy engaged by staple legs 120, head 114 is further retracted in a proximal direction indicated by arrow 440. Head 114 is proximally retracted until the distal end of head 114 is located proximally of protrusions 338, thus allowing legs 120 to move radially inward toward axis 326, gathering tissue and closing the arteriotomy as staple 102 attempts to resume the closed configuration.

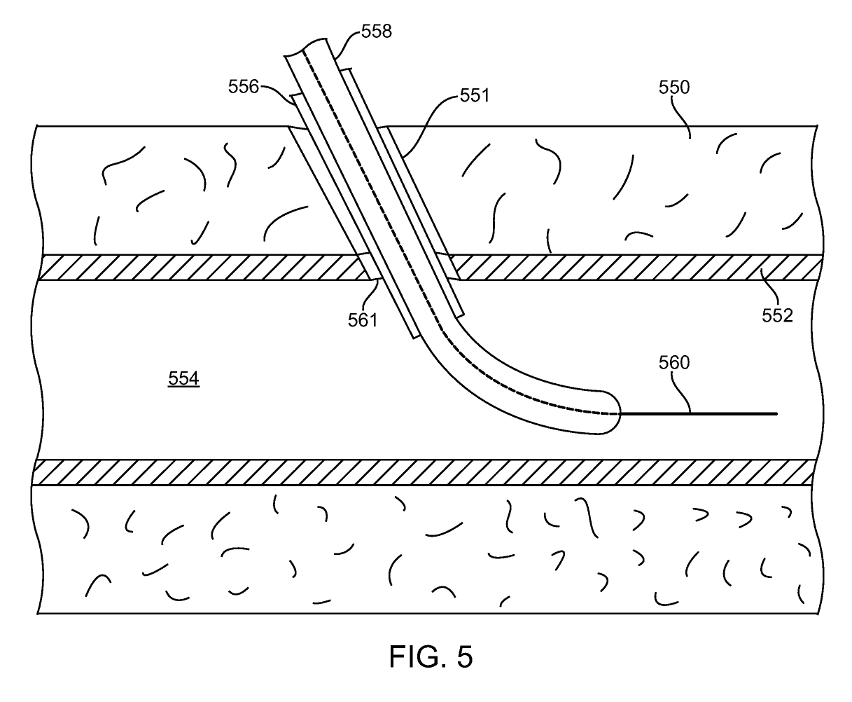

With reference now to FIGS. 5-11, an exemplary method for closing an opening after a procedure in which a vessel was punctured to gain access to the vessel lumen will be described utilizing the staple delivery system 100 described above. The method will be described with respect to closure of an opening in an artery, i.e. an arteriotomy. However, one of ordinary skill in the art will recognize that the same or similar procedure can be used to close openings in veins, i.e. a venipuncture, or to close openings in other vessels in the body of a patient. The staple delivery system includes a head having a plurality of channels configured to receive the prongs of corresponding staple legs so that the prongs and/or the surrounding tissue do not become damaged while the staple is being delivered through a tissue track to the region of the arteriotomy. The head channels also allow the stapling system to have a low delivery profile.

FIGS. 5-11 illustrate the staple delivery system advanced through the tissue track to the region of the arteriotomy without a delivery sheath and/or stabilizing device, as will be described in an alternative method below. The staple delivery system may be advanced through a tissue track without a delivery sheath since the prongs of the staple legs are housed within the channels of the mandrel head. The channels guard or protect the prongs so that they do not become unintentionally engaged with other objects or tissue along the tissue track. In addition, the generally tapered distal end of the staple delivery system facilitates advancement of the system by gently dilating the pre-formed tissue track as it is advanced therethrough. Alternatively, it will be understood by those of ordinary skill in the art that the invention may be practiced in conjunction with a delivery sheath and/or stabilizing device in order to facilitate tracking of the staple delivery system to the region of the arteriotomy. For example, the invention may be practiced in conjunction with a stabilizing device, for example, of the type described in U.S. Pat. No. 6,767,356 (Kanner), the entire disclosure of which is incorporated herein by reference. As described in the Kanner '356 patent, wire-like stabilizers extend distally from a delivery sheath. The distal ends of the stabilizers are configured to be placed through the arteriotomy into the lumen of a vessel. The stabilizers, which are inserted in a linear configuration, then are actuated into an enlarged configuration, so that they resist being withdrawn through the arteriotomy. The stabilizers and the delivery sheath provide a stable delivery platform held in a centered position over the region of the arteriotomy. The staple delivery system of the present invention may be inserted through the delivery sheath and advanced to the region of the arteriotomy. Reference is made to the Kanner '356 patent for additional details concerning various constructions and embodiments of the delivery sheath and stabilizing system, which are incorporated by reference herein, in their entirety.

FIGS. 5-8 show sectional views of body tissue 550 with a vessel 552 disposed therein. FIG. 5 shows a procedural device 558, such as a catheter, riding along a guidewire 560 and being disposed partially within a lumen 554 of vessel 552. Procedural device 558 extends through an introducer sheath 556, which was used during the procedure to maintain access to lumen 554 through a tissue track 551 in tissue 550 and arteriotomy 561 in the wall of vessel 552. Tissue track 551 is shown as being larger than sheath 556 for convenience of illustration. In practice, introducer sheath 556 is snugly fitted within, and may have been used to form tissue track 551 and arteriotomy 561.

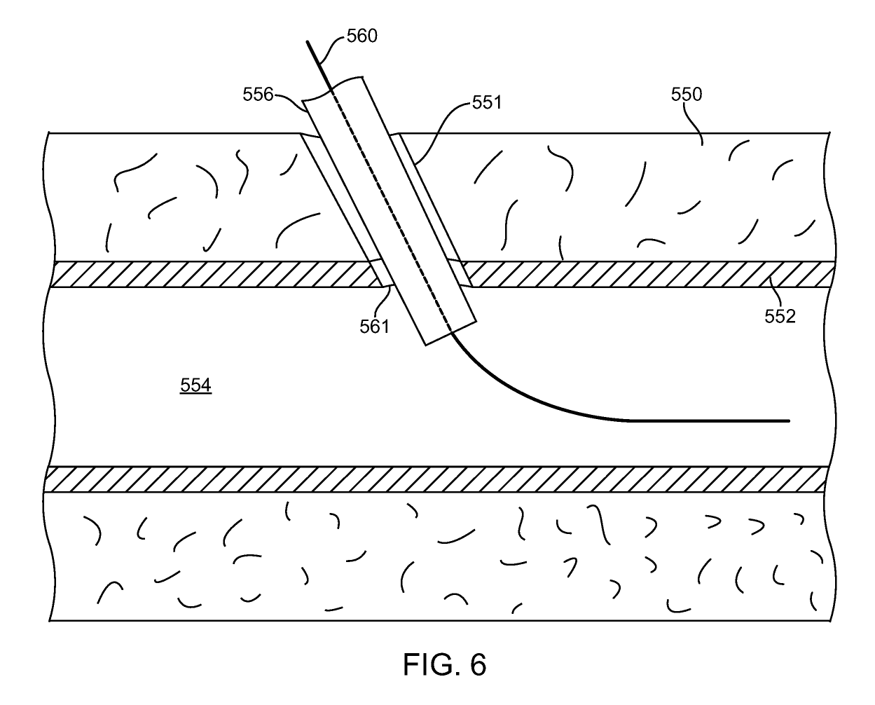

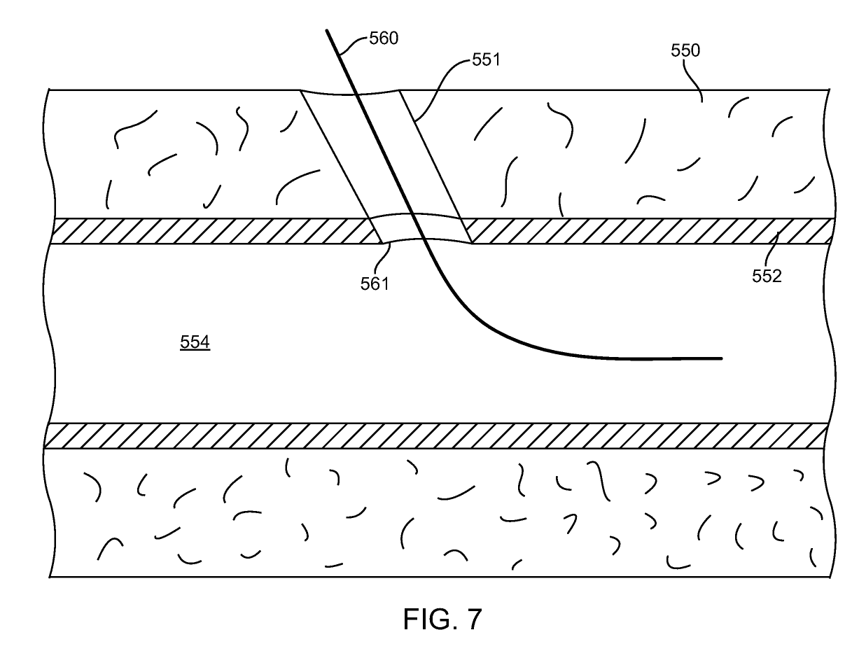

At the end of the catheterization procedure, procedural device 558 is removed from vessel 552 and tissue track 551 via introducer sheath 556, as shown in FIG. 6. Guidewire 560 remains in place partially disposed within lumen 554 of vessel 552. As shown in FIG. 7, introducer sheath 556 is removed from lumen 554, arteriotomy 561, and tissue track 551, leaving guidewire 560 in place. Although tissue track 551 and arteriotomy 561 are shown in FIG. 7 as being maintained in an open configuration, in practice, tissue track 551 and arteriotomy 561 will tend to at least partially close around guidewire 560 when introducer sheath 556 is removed.

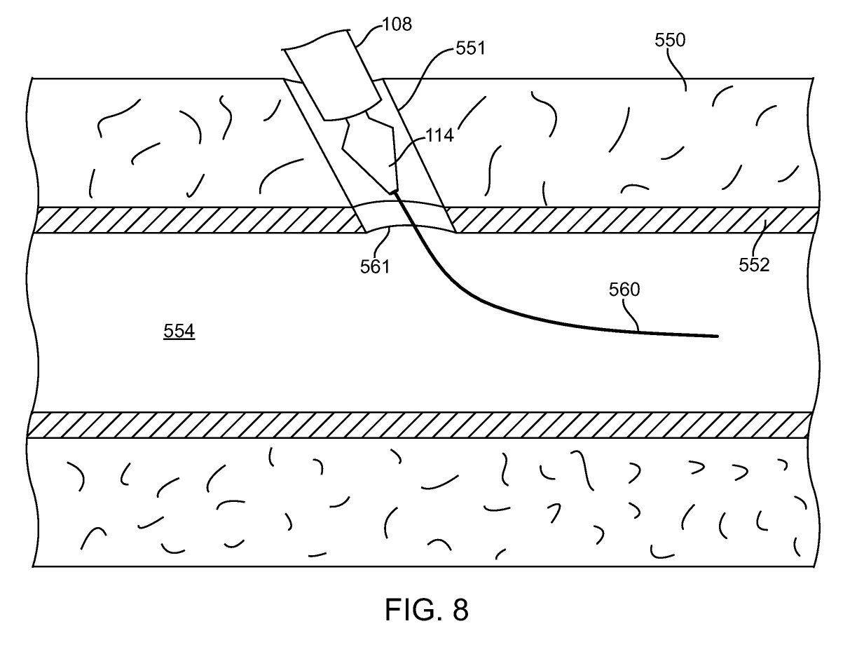

Staple delivery system 100 of the present disclosure is used to close arteriotomy 561 as follows. As described above, staple delivery system 100 includes mandrel 112 with head 114 at the distal end thereof carried slidably within sleeve 108. Head portion 114 is responsible for generating the expansion forces on the staple, and further includes a plurality of channels for guarding the distal portion or sharp tips of the staple legs. As shown in FIG. 8, staple delivery system 100 is guided along guidewire 560 into tissue track 551. As described with respect to FIGS. 5 and 6 above, tissue track 551 is shown as being larger than sleeve 108 and head 114 for convenience of illustration. In practice, sleeve 108 and head 114 are snugly fitted within tissue track 551, and head 114 may dilate or re-dilate tissue track 551 and arteriotomy 561 as it is inserted therethrough.

Although not visible on FIG. 8, the prongs of the staple legs are housed within channels 116 of mandrel head 114. Staple delivery system 100 rides along guidewire 560 to a location within tissue track 551 spaced proximally from vessel 552. In this embodiment, mandrel 112 of the staple delivery system is provided with a guidewire lumen (not shown) extending therethrough such that system 100 may be loaded onto and advanced along indwelling guidewire 560. Guidewire 560 directs the staple delivery system through tissue track 551 to a centered position over the region of arteriotomy 561. However, it will be understood by those of ordinary skill in the art that staple delivery system 100 may be delivered to the region of arteriotomy 561 without the use of a guidewire. Rather, guidewire 560 may be removed from the tissue track prior to insertion of the staple delivery system. With the prongs of the staple legs housed within the channels of the mandrel head, the generally tapered distal end of mandrel head 114 of staple delivery system 100 facilitates advancement of the system to the region of the arteriotomy.

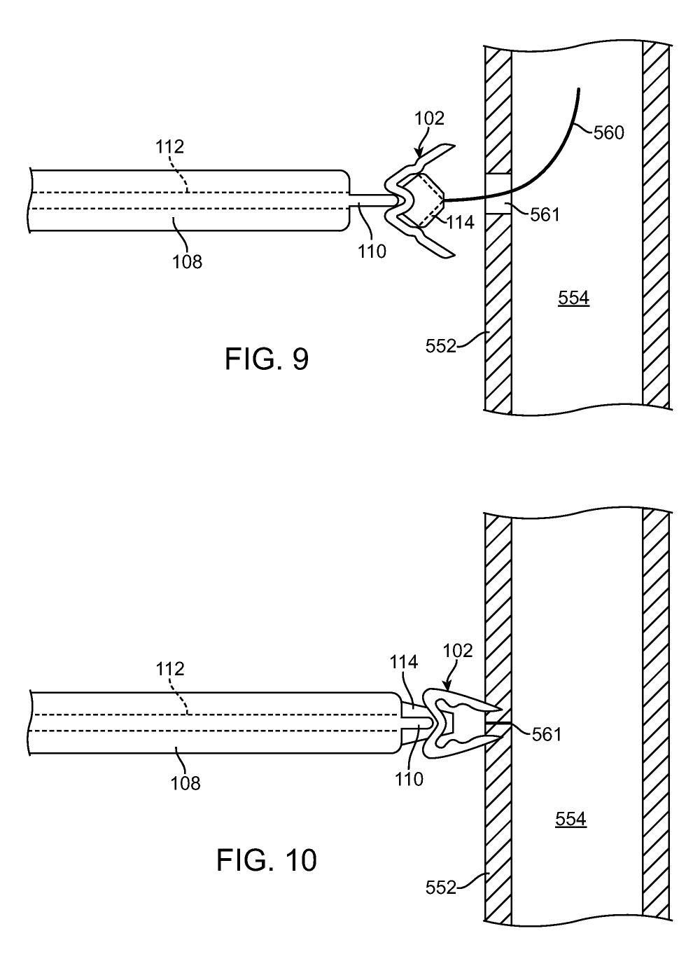

FIGS. 9-11 illustrate side views in which vessel 552 is shown in longitudinal cross-section and tissue 550 has been excluded for convenience of illustration. The distal end of staple delivery system 100 is located extraluminally, proximal to arteriotomy 561. As shown in FIG. 9, head 114 is proximally retracted in order to expand the staple legs of staple 102 as described above. Simultaneously, or closely coordinated with the spreading of the staple legs, staple 102 in the expanded configuration is advanced distally such that the prongs of the staple legs pierce vessel 552 to engage vessel tissue on either side of, or surrounding arteriotomy 561. The expanded distance between staple legs 102 more than spans arteriotomy 561, as shown with respect to staple 102 in FIG. 9. As described above, staple tissue stops 334 help prevent staple legs 120 from fully penetrating the wall of vessel 552 and entering lumen 554. Once the staple legs engage vessel tissue, guidewire 560 may be proximally withdrawn and removed from the body.

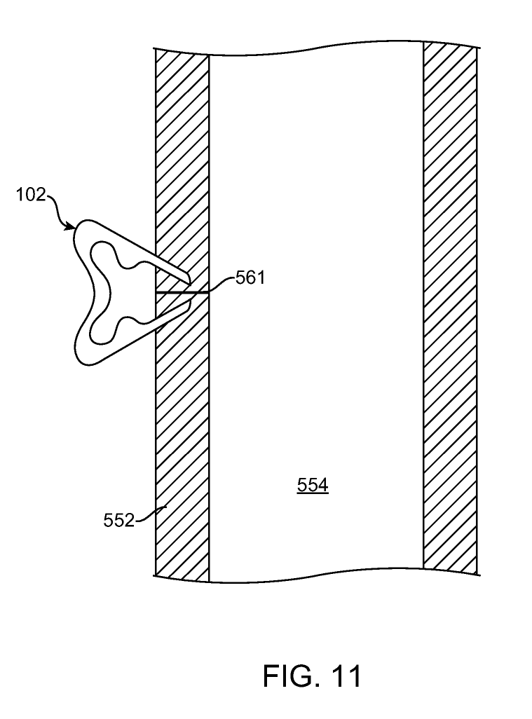

Referring now to FIG. 10, mandrel head 114 is withdrawn through the proximal end of staple 102 such that staple 102 is allowed to elastically revert towards the closed or unexpanded configuration as described above. As mandrel head 114 is proximally withdrawn, staple 102 is held stationary adjacent to arteriotomy 561 by fingers 110 of sleeve 108. As staple 102 closes, the staple legs gather or pucker the engaged tissue, thus closing arteriotomy 561. In the closed or unexpanded configuration, the distal tips of staple 102 may approach, or touch each other, depending upon the amount of tissue gathered between the prongs. Staple delivery system 100 is removed from the body while staple 102 remains embedded in vessel 552, as shown in FIG. 11.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A staple delivery system for closing a vascular puncture comprising:
a tissue staple including at least two staple legs, each staple leg having a distal prong terminating in a sharp distal tip; and
a stapler including an elongate mandrel having disposed at its distal end an enlarged head for expanding the at least two staple legs when the head is proximally withdrawn through the staple, the head including at least two opposing channels,
wherein the prongs are housed within the opposing channels prior to expansion of the staple legs.

2. The staple delivery system of claim 1, wherein the stapler further includes an outer sleeve adapted to slidably receive the mandrel therethrough.

3. The staple delivery system of claim 2, wherein a proximal end of the staple is supported by a distal end of the outer sleeve when the head is proximally withdrawn through the staple.

4. The staple delivery system of claim 1, wherein a proximal portion of the head includes a plurality of flared ramps for expanding the at least two staple legs.

5. The staple delivery system of claim 4, wherein a distal portion of the head includes a plurality of tapered surfaces.

6. The staple delivery system of claim 1, wherein the channels extend from a proximal end of the head to a distal end of the head.

7. The staple delivery system of claim 1, wherein a width of the channels is sufficient to house the prongs.

8. The staple delivery system of claim 1, wherein a depth of each of the channels is sufficient to completely house the corresponding prong to substantially prevent contact between the tips and a surrounding tissue track as the system is advanced to the region of the vascular puncture.

9. The staple delivery system of claim 1, wherein the staple has only two staple legs and the head has two corresponding channels for housing the prongs.

10. The staple delivery system of claim 1, wherein the staple is formed of a resilient metal such that the staple tends to revert back to an unexpanded configuration once the head is withdrawn from the staple.

11. A stapler for closing a vascular puncture comprising:
an outer sleeve;
a mandrel slidably received in the outer sleeve; and
a head disposed at a distal end of the mandrel, wherein the head is configured to expand legs of a staple when the head is proximally withdrawn through the staple,
wherein the head includes at least two opposing channels, each channel adapted for housing a distal tip of the staple leg prior to expansion thereof.

12. The stapler of claim 11, wherein a proximal portion of the head includes a plurality of flared ramps.

13. The stapler of claim 12, wherein a distal portion of the head includes a plurality of tapered surfaces.

14. The stapler of claim 11, wherein the channels extend from a proximal end to a distal end of the head.

15. The stapler of claim 11, wherein a depth of each channel is sufficient to completely house the corresponding prong to substantially prevent contact between the tips and the surrounding tissue as the staple is tracked to the region of the vessel puncture.

16. A method of closing a vessel puncture, the method comprising the steps of:
providing a stapler including a flared head for expanding staple legs of a staple when the head is proximally withdrawn through the staple, wherein the head includes at least two opposing channels;
providing a tissue staple including at least two staple legs, each staple leg having a distal prong terminating distally in a sharp tip;
positioning the tissue staple over the head such that at least the prongs are housed within the at least two opposing channels of the head;
advancing the head and the staple to the region of the arteriotomy;
proximally withdrawing the head through the staple in order to splay the staple legs, thereby radially separating the tips;
piercing tissue around the arteriotomy with the radially separated tips; and
allowing the prongs to resiliently close radially inwardly such that the staple closes around the vessel puncture.

17. The method of claim 16, wherein a proximal portion of the head includes a plurality of flared ramps for splaying the staple legs.

18. The method of claim 16, wherein the staple is formed of a resilient metal and the step of allowing the prongs to resiliently close includes proximally withdrawing the head from the staple such that the staple tends to revert to an unexpanded configuration.
